(19) United States
(12) Patent Application Publication
(10) Pub. No.: US 2021/0375429 A1
(43) Pub. Date: Dec. 2, 2021

(54) AUTOMATION OF MEDICAL NUTRITION THERAPY

(71) Applicant: Nutrimedy, Brookline, MA (US)

(72) Inventors: Mallory P. Franklin, Minneapolis, MN (US); Karolina Starczak, Beverly, MA (US)

(21) Appl. No.: 16/948,952

(22) Filed: Oct. 7, 2020

Related U.S. Application Data
(60) Provisional application No. 63/031,516, filed on May 28, 2020.
(60) Provisional application No. 63/031,513, filed on May 28, 2020.

(51) Int. Cl. G16H 20/60; G16H 10/60; G16H 50/20; G16H 50/70; G16H 70/20; G16H 40/67; G16H 40/20; G06Q 10/10; A61B 5/00; A61B 5/145

(52) U.S. Cl. CPC G16H 20/60; G16H 10/60; G16H 50/20; G16H 50/70; G16H 70/20; G16H 40/67; A61B 5/14532; G06Q 10/1095; A61B 5/4836; A61B 5/4833; A61B 5/742; A61B 5/7465; A61B 5/681; G16H 40/20

(57) ABSTRACT

A system and digital health platform that allows a healthcare provider to automate the delivery of medical nutrition therapy. Following the nutrition care process, the system obtains the patient's medical information and determines appropriate nutrition diagnosis, nutrition therapies, and what data should be collected and monitored for each patient. The system is used by registered dietitian nutritionists or other healthcare providers. Based on the patient's nutrition assessment, a frequency of interactions with a medical provider is identified for optimal outcomes. Using identified monitoring tools, patients are monitored for changes in their condition and appropriate nutrition interventions are updated based on these changes. The tool facilitates the evolution of nutrition research by data analytics regarding intervention, patient data, and outcome data.

### RELATED APPLICATION

The present application claims the benefit of U.S. Provisional Application Nos. 63/031,516 and 63/031,513, filed May 28, 2020, which are hereby incorporated herein in their entireties by reference.

### TECHNICAL FIELD

The technology relates to the general field of healthcare and has certain specific applications for registered dietitian nutritionists for medical nutrition therapy.

### BACKGROUND

Nutrition is an area of growing interest for patients, payors, and providers, since addressing patient nutrition care plays a critical role in disease management. With 60% of Americans managing at least one chronic condition and 40% managing multiple conditions, nutrition support is an increasing concern. Medical nutrition therapy is the process of “nutritional diagnostic, therapy, and counseling services for the purpose of disease management” and is delivered by a registered dietitian nutritionist or nutrition professional. To deliver medical nutrition therapy, registered dietitian nutritionists use the nutrition care process which consists of four steps: assessment, diagnosis (PES statement), intervention, and monitor and evaluate.

Medical Nutrition Therapy caters to the complications that arise due to multiple conditions with conflicting nutritional requirements, medications that interact with diet, changes in appetite, hyper- and hypo-metabolic states, damage to the gastrointestinal tract or NPO status, changes to taste during treatments, and dysphagia, the inability to chew and/or swallow. Medical Nutrition Therapy includes but is not limited to total parenteral nutrition, enteral nutrition and food or nutrient guidelines for the treatment, management or prevention of disease. For effective dietary alterations, registered dietitian nutritionists cater to the cultural, behavioral, and socioeconomic challenges to design personalized dietary modifications. Registered dietitian nutritionists are typically part of the care team and are confined to clinical or private practice settings. There are a little under 104,000 registered dietitian nutritionists in the United States. With roughly 133,000,000 Americans with one or more chronic conditions that would require dietary modifications, there is one registered dietitian nutritionist for every 1,279 patients. Therefore, there is a need to improve efficiency in how medical nutrition therapy is delivered while simultaneously increasing access, frequency, and personalization. Current medical nutrition therapy protocols require patients to schedule in-person visits with a registered dietitian nutritionist. Assessment, the first step of the nutrition care process, includes gathering patient medical history, allergies, medication, medical diagnoses, and dietary intake. Based on the information collected, registered dietitian nutritionists identify a nutritional diagnosis and develop an intervention for the patient often referencing nutrition guidelines and performing calculations by hand. Interventions include education materials, generalized handouts, and examples of meal plans. The patient is expected to incorporate these generalized examples of the needed dietary modifications on their own with no daily guidance. As such, patients are often unsuccessful in making lasting dietary changes to improve their condition or manage their disease.

Thus, there is a present need in the art for patient-facing tools to provide patient support between provider visits or options for virtual visits with the provider as needed. There is a further need in the art for automation of portions of the medical nutrition therapy process to relieve the burdens on providers.

### SUMMARY

Many physicians and registered dietitian nutritionists are seeking solutions to optimize efficiency and provide patient-facing tools to support in between visits or provide options for virtual visits. The present disclosure provides a tool to automate several of the steps for medical nutrition therapy. Through the growing field of telehealth and artificial intelligence, medical nutrition therapy could be more accessible for the patients that need it. This is especially important for rural patients who are often excluded from clinical medical nutrition therapy services due to distance and limited staffing at smaller local facilities.

The present disclosure outlines methods for automating the delivery of patient-tailored medical nutrition therapy through a digital health platform. The digital platform facilitates all stages of the nutrition care process to deliver medical nutrition therapy for any patient with any condition(s). The digital platform includes a patient-facing platform and a provider-facing platform that are linked, allowing providers to oversee the system's automated recommendations while also facilitating interaction between the provider and the patient. Through patient data collection the system identifies the nutrition diagnosis, identifies appropriate nutrition interventions based on medical nutrition therapy evidence-based protocols, and monitors the patients through a series of features built into the platform including video consults, customizable trackers, and a photo meal log that quantifies key nutrients.

Data can flow into the platform through integration with the electronic medical records, patient completion of a health bio survey, or entry by a medical provider, such as a registered dietitian nutritionist. Data is applied to conditional logic based on parameters to classify the collected data inputs into specific rules using algorithms and calculations. Rules are generated from evidence-based standardized medical nutrition therapy guidelines. Taken together the system generates individualized interventions guidelines for any condition or combination of conditions outlined.

Using these rules the system will automatically generate a nutrition related diagnosis, a PES statement: Problem, Etiology, Signs and Symptoms, as well as a set of interventions including nutrient recommendations and recommended frequency of visits with a registered dietitian nutritionist. These results are provided to the dietitian on the provider-facing interface. The patient will receive guidance on their nutrient intervention on the patient-facing platform in the form of educational handouts and resources from a database. The system automatically generates appointments based on patient and registered dietitian nutritionist availability. The system then produces a list of trackers that should be monitored for the patient, by creating trackers or suggesting integration to devices such as smart watches, scales, glucose monitors, and many more. On the patient-facing digital platform, the patient is prompted to enter meals using a photo meal log tracker and provides instantaneous feedback and visual cues regarding nutrient intake. The provider has insight into how the patient is doing from the trackers, integration, and data from the photo meal log.

Together these features increase efficiency for practicing registered dietitian nutritionists and provide a way to improve access to medical nutrition therapy for patients. The system then generates a provider note that is transmitted back to the patient's electronic medical record for billing purposes. The system is self-correcting as updates are made to electronic medical records and provider/patient input to adjust nutrient recommendations as conditions improve or worsen or additional diagnoses are identified.

Using data analytics the system will assess interventions and patient results to continue to adjust intervention protocols through machine learning technologies to optimize patient outcomes. As such, the system will continue to identify dietary interventions that have been proven effective for patients with similar demographics. This is a vital aspect for advancing the field of nutrition science and enhancing care protocols to improve health and outcomes.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 illustrates nutrition assessment relevant patient data collection.

FIG. 2 illustrates how the system generates nutrition diagnosis, appropriate interventions, and selects monitoring tools.

FIG. 3 illustrates how patient records and input continue to update the patient's recommended interventions.

While various embodiments are amenable to various modifications and alternative forms, specifics thereof have been shown by way of example in the drawings and will be described in detail. It should be understood, however, that the intention is not to limit the claims to the particular embodiments described. On the contrary, the intention is to cover all modifications, equivalents, and alternatives falling within the spirit and scope of the subject matter as defined by the claims.

### DETAILED DESCRIPTION OF THE DRAWINGS

The present disclosure outlines methodologies and systems for delivering medical nutrition therapy remotely via a digital platform. By using a customized digital platform, patients can engage in continual medical nutrition therapy support to meet their needs and level of understanding.

The system and digital platform provide a unique patient experience that may accommodate all modes of medical nutrition therapy delivery for any patient managing any condition(s). Patients visiting a physician to diagnose or address a medical condition can be referred to the digital tool, where they can create an account and may be prompted to complete a health bio survey to facilitate data collection. The attending registered dietitian nutritionist or other provider can log into the digital tool and access the patient's account, inputting any additional data needed for assessment. Patient data from the electronic medical record can feed into the digital tool. Together these data entries can be used by the system to provide the attending registered dietitian with a set of automated diagnosis, interventions, and patient metrics to be monitored. The patient can automatically receive nutrient recommendations, appropriate handouts and educational materials, and a list of trackers that can either be integrated to devices they possess or entered manually. Patients may be prompted to enter trackers and log meals, and may receive automated or requested feedback based on their unique intervention. Data entered by a patient can be viewed by their registered dietitian nutritionist or provider. Based on the patient's diagnosis, intervention, and inputs into trackers for monitoring, the digital tool can automatically recommend a frequency of virtual visits with the registered dietitian nutritionist. Based on both the availability of the registered dietitian nutritionist and the patient, appointments may be automatically scheduled, with requests sent to each party. The virtual sessions may take place over the platform and during the visits data from trackers, meals, and generated recommendations from the system can be reviewed to facilitate the conversation between the patient and the registered dietitian nutritionist. Allowing the system to set the frequency and cadence of virtual appointments can help ensure that patients with the biggest need receive more care, while those who are doing well may not need a follow up for many months. This approach allows for efforts and staffing to be focused to optimize outcomes and efficiency.

### Automating Medical Nutrition Therapy:

Aspects of the present disclosure automate medical nutrition therapy, a service that is currently manual, time intensive, and provided by registered dietitian nutritionists often only in a clinical setting.

The first step of the nutrition care process is assessment. By creating robust tools for capturing patient data, the system avoids human error that could miss critical aspects of the patient's medical information. By getting a full picture of the patient through the many avenues for data collection, the system can optimize and prioritize patient needs.

The second step of the nutrition care process is diagnosis. Nutrition diagnosis may focus on the most critical nutrition concern for the patient, although many may be present. In embodiments, all diagnoses may be presented, prominence may be given based on severity, immediacy, or any number of factors. The diagnosis may be presented broken into a PES statement: Problem, Etiology, Signs and Symptoms. The system generates a PES statement, for example, from a database of terminology used for medical nutrition therapy.

The next step of the nutrition care process is the intervention. Nutrition recommendations for managing chronic conditions can differ based on the condition(s). For example, a patient with chronic kidney disease may need to reduce protein intake, while a patient with sarcopenia would need to increase protein intake. In many instances' patients have multiple conditions leading to complicated recommendations for optimal nutrition. In such cases, the system may not only provide condition-specific nutrition interventions but can reason between conflicting nutrition recommendations depending on the patient's many conditions. By doing so, the system can draw ideal nutrition requirements to fit the patient's unique needs. Nutritional guidance may then automatically be provided through educational materials, handouts, recipes, and modules through the patient-facing digital platform. In addition, based on the complexity, severity, or level of perceived patient knowledge, the system may recommend a frequency of virtual appointments between the patient and the registered dietitian nutritionist. The system can connect to the patient's and the provider's calendar to determine times at which both the patient and the provider are available. The system can automatically schedule recurring appointments based on frequency and cadence recommended. This increases efficiency since most follow ups for medical nutrition therapy are set to review the patient's progress rather than being based on the patient's progress.

The final step of the nutrition care process is monitor. Depending on the patient's condition or intervention, a set of trackers are used to monitor the patient's progress. These may be identified in the system based on the interventions selected. Trackers for manual entry are generated or where appropriate the system prompts for an integration with an existing device.

In another aspect of the disclosure in which a patient requires enteral or parenteral nutrition therapy, the system can generate recommendations based on the collected patient data. Utilizing a database of nutrient content from enteral and parenteral products, the system will recommend a brand, flow rate, total volume and outline other variables necessary for patient success as it pertains to medical nutrition therapy.

Embodiments of the present disclosure may identify trackers to monitor the patient, which would be indicative of how the patient is doing. Utilizing these trackers, which can include but are not limited to manual entry, integration from medical devices that sense vitals, scales, wearable devices, changes to laboratory results, or updates to their medication, recommendations can be updated or reinforced based on fluctuation in patients' needs. For example, if a patient's weight suddenly fluctuates, an indication of edema or fluid retention, the patient will receive an alert to limit sodium and fluid until the edema is resolved, preventing further complications. In another instance, changes to medication, such as a diuretic, could change fluid or electrolyte restrictions. Patients who are admitted to the hospital, updates to their medical records can feed directly into the system, which may automatically lead to adjustments based on the hospital visit. As the patient's medical record is updated, trackers are entered, and meals are logged, the system can adjust daily recommendations to meet the patients changing needs. The system can also set a threshold of alarm, in which a registered dietitian nutritionist or other medical provider may be alerted about a possible concern due to thresholds set for each tracker.

### Healthcare Provider User:

The present disclosure can be used by medical providers, predominantly registered dietitian nutritionists to increase reach and access to medical nutrition therapy services. The platform automates a majority of the initial assessment, calculations, and interventions performed by a healthcare provider. Using the healthcare provider interface, registered dietitian nutritionists, and other medical staff, can view patient information regarding meals consumed, nutrients consumed and review analysis of how the patient is doing with their dietary modifications. Based on these metrics the system can generate alerts for patients who are struggling with adherence and allow medical providers to address the needs of patients who need further support. By doing so, the system may be configured to prioritize patients based on need and staff capacity. This may improve outcomes in a health system by not wasting resources on patients who are successfully maintaining their nutrient intake appropriately. The system may also improve efficiency by generating a note for billing medical nutrition therapy that is fed back into the electronic medical record.

### Patient Users:

The present disclosure describes a digital tool that patients can use to receive personalized-nutrition guidance. Patients can engage with the platform to review educational materials or learn about how nutrition plays an important role in the management of their condition. Nutrient recommendations may be outlined based on these education materials or using a photo meal log, patients are able to track their intake of the key nutrients. By receiving feedback about their intake and how it compares to recommendations, patients are guided on a day to day basis regarding their nutrition approach. Patients may receive not only daily guidance around dietary choices and oversight by the system using the trackers and integrations, but they can also connect with their dietitian remotely through instant messaging and virtual appointments, allowing patients to receive nutrition counseling whenever and wherever is convenient for them. A patient in a rural town will now be able to consult with their dietitian without having to schedule an appointment, take time off work, or drive the far distance to the hospital.

In another embodiment of the present disclosure a patient that has received generated nutrition recommendations and is uploading meals using the photo meal log feature, the system can predict unwanted outcomes such as increased blood pressure, weight gain, or hyperkalemia due to dietary behaviors. For example, a patient with end stage renal disease who is going to consume a large spinach salad will be notified of the risk of hyperkalemia due to that meal.

### Data Analytics and Progression of Nutrition Care:

Nutrition is an evolving area of research that faces many challenges in identifying nutrition standards for condition management. Using a systematic approach to medical nutrition therapy allows for the collection of detailed nutrition information and correlating outcome data. Through machine learning the system will gather patient data and identify the most effective intervention steps that have led to the desired outcomes across conditions, genders, age, and other demographic data.

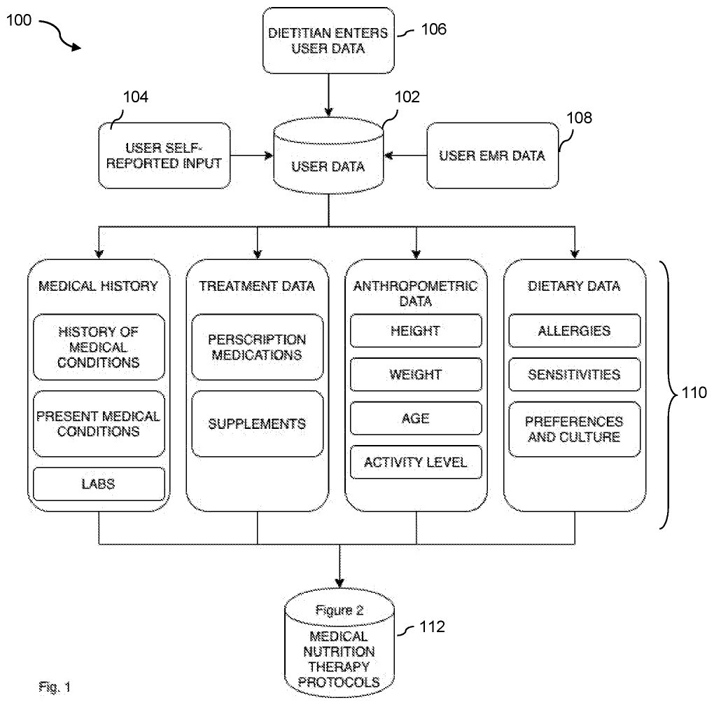

Referring now to FIG. 1, a workflow 100 of nutrition assessment relevant patient data collection is shown. User data 102 may be assembled from multiple possible sources, such as self-reporting 104, provider reporting 106, or retrieval from an electronic medical record 108. Other possible sources of user data are envisioned, such as trackers, which are not depicted here. User data 102 may be categorized 110 to improve management, organization, and ease of utility by the system. Categories may vary according to a particular user's needs or preferences, or may be a default setting. Categorized or otherwise processed user data 110 may then be used by the system to develop recommended nutrition therapy protocols 112.

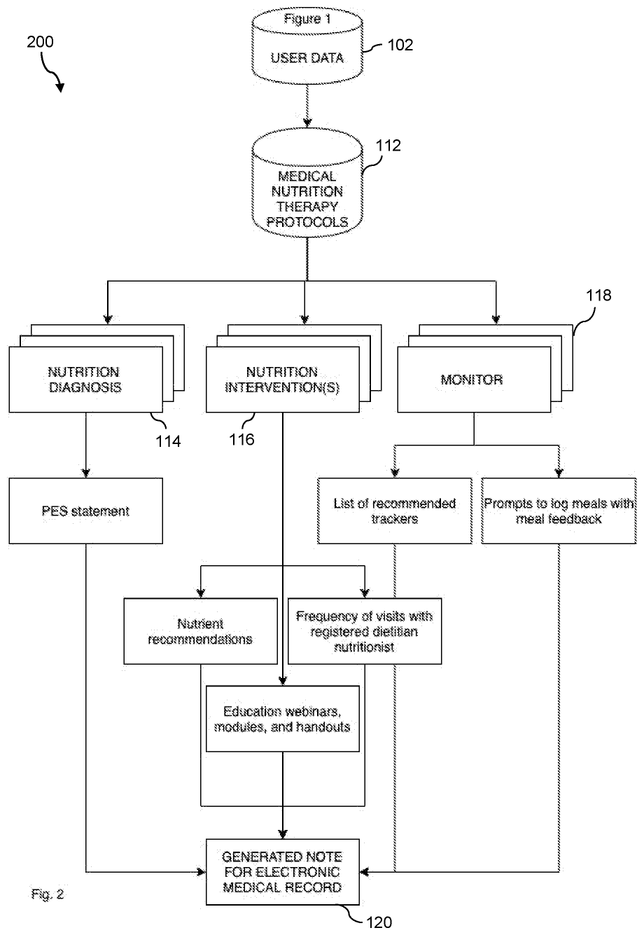

Referring now to FIG. 2, a workflow 200 demonstrates how the system generates nutrition diagnosis, appropriate interventions, and selects monitoring tools. In embodiments, therapy protocols 112 may be broken out into diagnosis 114, intervention(s) 116, and monitoring 118. Diagnosis 114, intervention 116, and monitoring 118 may proceed in parallel, or in sequences of varying order. One or more may be missing or duplicated, or additional elements may be appended or used to replace one or more of those in this example. Each element of therapy protocol 112, diagnosis 114, intervention 116, and monitoring 118 in this example, may be integrated into a provider's note, which may be editable by the provider and automatically integrated with the user's electronic medical record 120. The user data 102 used and the processing performed by vary by element. Diagnosis 114 may generate a PES statement, containing Problem, Etiology, Signs and Symptoms. Interventions 116 may recommend a variety of possible options, including but not limited to nutrient recommendations, a particular frequency or periodicity of provider visits, additional education in the form of webinars, modules, or handouts which may be integrated with the system in embodiments, etc. Monitoring 118 may draw on sources used to generate user data 102 (such user surveys 104, provider input 106, or the user's electronic record 108) and may additionally recommend or draw upon other sources of user data, for example, by recommended trackers or prompting a user to use a meal log. All are combined into a proposed provider's note 120 to be reviewed by the provider, and entered into the user's electronic medical record once edited and approved by the provider.

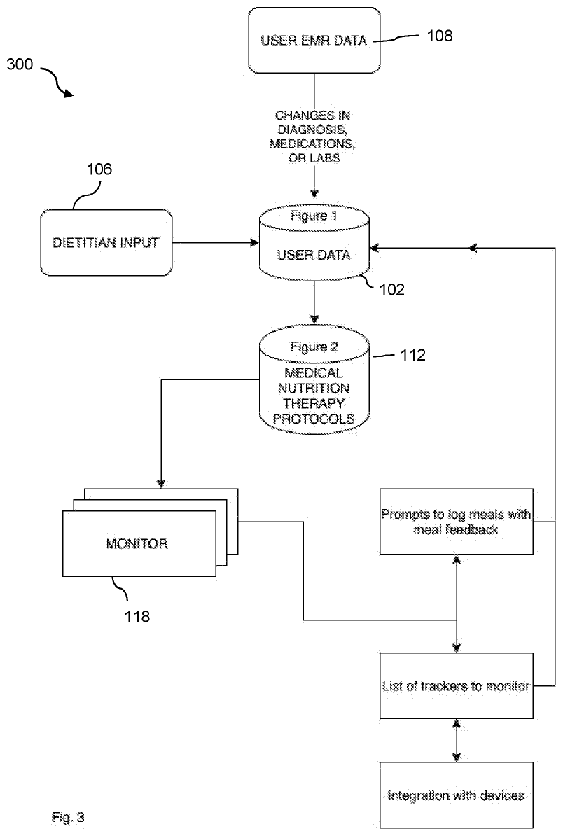

Referring now to FIG. 3, workflow 300 illustrates how patient records and input continue to update the patient's recommended interventions. User data 102 and therapy protocols 112 were described in greater detail above in conjunction with FIG. 1 and FIG. 2, respectively. In flow 300, the feedback of monitoring 118 to update user data 102 is shown, IN addition to being part of the recommendation in provider note 120 (shown in FIG. 2), monitoring 118 continually adds to and revises user data 102 to maintain a complete and accurate picture of the user's health to support accurate diagnosis and recommendation of effective therapies. Additionally, input from the provider 106 and the electronic medical record 108 may be used further than initially populating the user data 102. Additional provider input 106 and changes and additional to the electronic medical record 108 may be continually processed by the system and used to update user data 102.

Various embodiments of systems, devices, and methods have been described herein. These embodiments are given only by way of example and are not intended to limit the scope of the claims. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claims.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A digital platform for the delivery of medical nutrition therapy, comprising:
a patient-facing platform configured to receive data from integrated devices and a user in the form of a meal log tracker and a user electronic medical record and provide real-time feedback and at least one cue regarding nutrition intake; and
a provider-facing platform communicatively coupled with the patient-facing platform, and configured to receive data from the patient-facing platform;
wherein the digital platform generates a provider note to an electronic medical records system based on data from both the patient-facing platform and the provider-facing platform.

2. A method for the delivery of medical nutrition therapy, comprising:
collecting patient data;
identifying a nutrition diagnosis by classifying the patient data into rules, wherein the rules are generated from nutrition therapy guidelines;
identifying an appropriate nutrition intervention for the nutrition diagnosis based on medical nutrition therapy evidence-based protocols;
displaying the nutrition diagnosis, the nutrition intervention, and a Problem, Etiology, Signs and Symptoms (PES) statement to a provider-facing platform for review; and
monitoring the patient for compliance with the nutrition intervention.

3. The method of claim 2, wherein monitoring the patient comprises one or more of:
conducting a video consultation, using a customizable tracker, or implementing a photo meal log.

4. The method of claim 2, wherein collecting patient data comprises one or more of:
integrating the provider-facing platform with a patient electronic medical record, completing a survey by the patient, or entering the patient data by a medical provider.

5. The method of claim 2, wherein identifying an appropriate nutrition intervention includes:
identifying at least one nutrient recommendation; and
recommending a frequency of visits with a medical provider for the patient.

6. The method of claim 2, further comprising automatically generating an appointment according to both a patient availability and a provider availability.

7. The method of claim 2, wherein monitoring the patient and collecting patient data each comprise receiving data from an integrated device.

8. The method of claim 7, wherein the integrated device is one or more of: a smart watch, a smart phone, a wearable fitness tracker, a scale, or a glucose monitor.

9. The method of claim 7, wherein identifying an appropriate nutrition intervention comprises recommending one or more integrated devices.
